(19) United States
(12) Patent Application Publication
(10) Pub. No.: US 2011/0224629 A1
(43) Pub. Date: Sep. 15, 2011

(54) Inner Ear Drug Delivery Device and Method

(75) Inventors: Claude Jolly, Innsbruck (AT); Alessandro Martini, Podova (IT)

(73) Assignee: MED-EL ELEKTROMEDIZINISCHE GERAETE GMBH, Innsbruck (AT)

(21) Appl. No.: 13/105,220

(22) Filed: May 11, 2011

Related U.S. Application Data
(62) Division of application No. 12/579,778, filed on Oct. 15, 2009.
(60) Provisional application No. 61/105,493, filed on Oct. 15, 2008.
(60) Provisional application No. 61/180,999, filed on May 26, 2009.

(51) Int. Cl. A61M 31/00; A61M 5/00

(52) U.S. Cl. 604/272; 604/285

(57) ABSTRACT

A drug delivery device for the inner ear is described. A drug delivery member for the inner ear without any stimulation electrodes has an intra-cochlear portion that penetrates into the inner ear of the patient and contains a drug eluting polymer material having at least one therapeutic drug which is released over time in a therapeutically effective amount into fluid in the inner ear of the patient. An extra-cochlear portion of the drug delivery member resides in the middle ear of a patient and completely occludes where the intra-cochlear portion penetrates into the inner ear. An anchor rod is within the intra-cochlear portion of the drug delivery member and adapted to extend out into the middle ear, ending in a retrieval knob for pulling the drug delivery member out of the cochlea.

This application is a divisional application of U.S. patent application Ser. No. 12/579,778, filed Oct. 15, 2009, which in turn claims priority from U.S. Provisional Patent Application 61/105,493, filed Oct. 15, 2008, and from U.S. Provisional Patent Application 61/180,999, filed May 26, 2009, which are incorporated herein by reference.

### FIELD OF THE INVENTION

The present invention relates to medical implants, and more specifically to a drug delivery device for the inner ear.

### BACKGROUND ART

There are many inner ear disorders which can lead to some degree of hearing loss. Among these are sudden hearing loss, noise induced hearing loss, progressive hearing loss, aminoglycoside induced hearing loss, presbiyacusis etc., autoimmune inner ear disorder, and infections (bacterial, viral, fungal). Many of the diseases that lead to partial or total hearing loss could utilize a therapeutic pharmaceutical treatment to reach some tissue or cells within the inner ear, for example, to arrest or reverse the hearing loss and improve hearing. Examples of therapeutic pharmaceutical molecules include without limitation cortico-steroids, peptides, and other proteins.

But there are relatively few ways to deliver therapeutic drugs to the inner ear. Typical clinical practice involves either oral, veinous, or arterial drug delivery. Topical drug delivery to treat the inner ear is limited to deposition of the drug at the round window and relying on diffusion of the drug through the round window to reach targeted cells. This may be accomplished by flooding the middle ear cavity with the drug in liquid form, or by applying a soaked sponge at or near the round window, for example, through an opening in the tympanic membrane. But a diffusion process through the round window is not particularly predictable or reliable. The permeability of the round window varies greatly between patients and by other criteria such as time of day, physical conditions, application methods, drug used. Thus, the amount of drug that reaches the inner ear through such delivery methods may vary anywhere between zero and toxically too much.

### SUMMARY OF THE INVENTION

Embodiments of the present invention are directed to a drug delivery device for the inner ear. A drug delivery member for the inner ear without any stimulation electrodes has an intra-cochlear portion that penetrates into the inner ear of the patient and contains a drug eluting polymer material having at least one therapeutic drug which is released over time in a therapeutically effective amount into fluid in the inner ear of the patient. An extra-cochlear portion of the drug delivery member resides in the middle ear of a patient and completely occludes where the intra-cochlear portion penetrates into the inner ear. An anchor rod is within the intra-cochlear portion of the drug delivery member and adapted to extend out into the middle ear, ending in a retrieval knob for pulling the drug delivery member out of the cochlea.

In further specific embodiments, the intra-cochlear portion may have a cylindrical rod shape or a conical shape. The drug eluting polymer material may be a flexible polymer strip and/or a silicone material. The intra-cochlear portion may penetrate into the inner ear through the round window or through a cochleostomy opening. The therapeutic drug may be incorporated into the drug eluting polymer material and/or may be a coating on the surface of the drug eluting polymer material.

Some embodiments may also include a support wire within at least a portion of the drug delivery device to provide supporting stability to the drug delivery member. In such an embodiment, the support wire may further include a puncturing point towards an apical end of the intra-cochlear portion for puncturing the round window membrane to insert the intra-cochlear portion into the inner ear.

### BRIEF DESCRIPTION OF THE DRAWINGS

FIG. 1 shows anatomical structures of a typical human ear having a drug eluting rod in the inner ear according to one specific embodiment of the present invention.

FIG. 2 shows a portion of another embodiment having an intra-cochlear section and an extra-cochlear section.

FIG. 3 illustrates another embodiment which fully occludes the opening into the inner ear.

FIG. 4 shows an example of an embodiment having an internal supporting wire.

FIG. 5 illustrates an embodiment having a sharp metallic tip.

FIG. 6 shows an example of an embodiment having an internal anchor rod and retrieval knob.

FIG. 7 shows an embodiment as in FIG. 6 in position in the ear of a patient.

FIG. 8 shows a drug delivery catheter arrangement according to another embodiment of the present invention.

### DETAILED DESCRIPTION OF SPECIFIC EMBODIMENTS

Embodiments of the present invention are directed to a drug delivery device for insertion into the inner ear of a patient. Such a device is supported by recent advances in cochlear implant technology with regards to surgery and electrodes that preserve hearing. If proper surgical techniques and equipment are used, the inner ear can be entered either through the round window membrane or a cochleostomy without causing excessive trauma. It has been shown (both in animal testing and in human patient use) that appropriate placement of a cochlear implant electrode through the round window membrane over a limited distance into the scala tympani does not severely interfere with the mechanical functioning of the inner ear. Such drug delivery devices are introduced with minimum trauma through the round window membrane or cochleostomy.

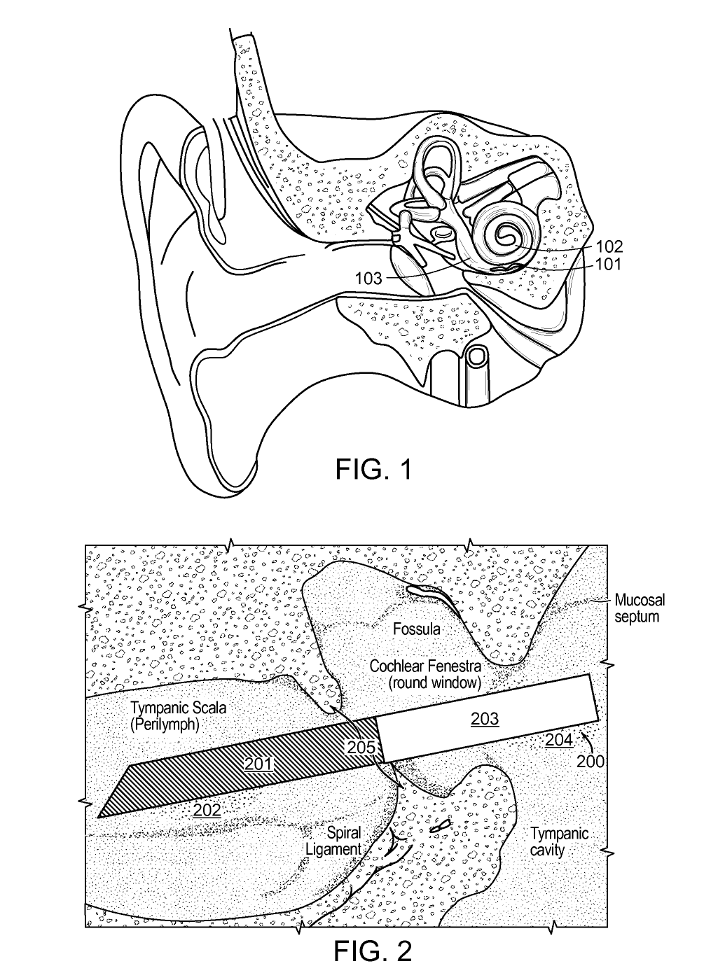

For example, as shown in FIG. 1, an intra-cochlear portion of a drug delivery member 101 completely penetrates into the inner ear (cochlea) 102 of the patient and contains a drug eluting polymer material which is impregnated and/or coated with one or more therapeutic drugs (e.g., dexamethasone) that are released in therapeutically effective amounts over time directly into fluid in the inner ear of the patient. And unlike a cochlear implant electrode, there are no stimulation electrodes on the drug delivery member 101. The intra-cochlear portion of the drug delivery member 101 and the drug eluting polymer material may be in the general shape of a cylindrical rod or conical shaped. Mechanisms for elution of the therapeutic drug from the drug eluting polymer material into an aqueous solution are known and reproducible. Different drug dosages can be achieved by varying the drug eluting polymer materials and their drug loading.

The drug delivery member 101 can be inserted into the inner ear 102 through a surgically created opening in the round window 103 or through a cochleostomy through the sidewall of the inner ear 102. The drug delivery member 101 can be inserted through the round window 103 from the outer ear canal and a tympano-meatal flap, for example, using robotic surgery with a key hole approach. Alternatively, a suprameatal approach, an atticotomy approach, a trans-canal approach, a mastoidectomy and posterior tympanotomy approach, or a myringothomy approach for insertion of the drug delivery member 101. When the round window 103 is not in a direct line with the tympanic membrane opening, a curved tubular or semi-tubular insertion instrument may be used to guide the drug delivery member 101 into or toward the round window 103. The additional advantage of a curved insertion instrument is that it offers a rigid guide for a flexible drug delivery member 101 to be inserted into the inner ear through the round window 103.

The drug delivery member 101 can remain permanently within the inner ear 102—cochlear implant electrodes have been left in place for many years after initial implantation without complications. But in those cases where there is subsequently further hearing loss, the drug delivery member 101 can be removed (e.g., via the round window 103). Then later another new drug delivery member 101 could be used, either with the same drug or with a more potent drug, with same concentration and dosage, or different concentration or dosage.

FIG. 2 illustrates a portion of another embodiment of a drug delivery device 200 having a cylindrical rod-shaped intra-cochlear drug delivery member 201 having a diameter of 0.3 to 1.0 mm that is positioned within the cochlea 202 (inner ear), and another extra-cochlear section 203 that remains in the middle ear 204. The intra-cochlear drug delivery member 201 is adapted to be inserted through the round window membrane 205 or through a cochleostomy drilled on the promontory. The insertion depth of the intra-cochlear drug delivery member 201 may be as great as 25 mm, but preferably is around 8 mm, which is the approximate length of the straight portion of the cochlea 202 before the intra-cochlear drug delivery member 201 would hit the outer wall of cochlea 202. The intra-cochlear drug delivery member 201 and the extra-cochlear section 203 typically are substantially smaller than the round window membrane 205 to avoid interference with the mechanical movement of the membrane.

The intra-cochlear drug delivery member 201 that enters the cochlea 202 includes a drug eluting polymer material which includes a concentration of a therapeutic drug, either in the form of a surface coating or as particles or crystals that are mixed and interspersed within the polymer material so that the therapeutic drug can be slowly released from the intra-cochlear drug delivery member 201 over time in the surrounding aqueous solution (i.e., the inner ear fluids). The concentration loading of the therapeutic drug may be anywhere between 0.1 and 20% weight of drug particles in the drug eluting polymer material of the intra-cochlear drug delivery member 201. Specific device arrangements and different drug loading and release profiles can be tailored to fit specific patient conditions and specific treatment requirements. Drug release can occur over a few weeks up to a several years depending on the drug loading and/or coating.

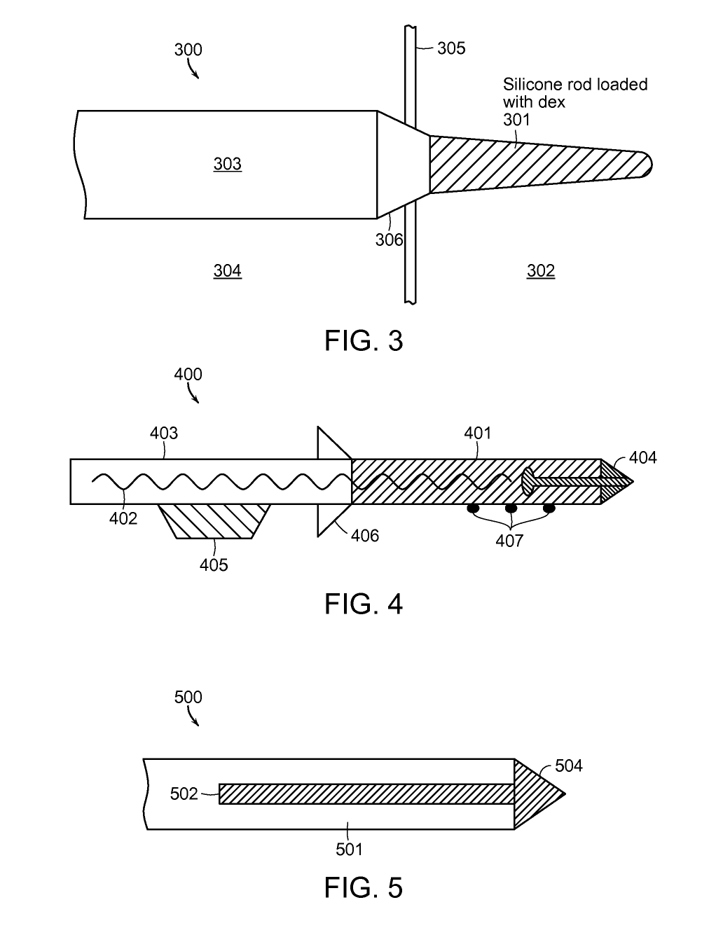

FIG. 3 illustrates another embodiment of a drug delivery device 300 in the form of a generally cylindrical silicone polymer rod which fully occludes the opening into the cochlea 302. The polymer rod has a tapered section 306 that when inserted fits snuggly in the opening to the round window membrane 305. A tight fit minimizes fluid leakage from the inner ear 302 to the middle ear 304 and promotes rapid healing around the device at the round window membrane. Other embodiments may have a similar tapered section that would fit snuggly into a cochleostomy opening. An intra-cochlear drug delivery member 301 contains the therapeutic drug which is released over time into the inner ear fluids within the cochlea 302 The extra-cochlear portion 303 that remains in the middle ear 304 may be made of a non-eluting polymer material to avoid unwanted delivery of medicine to the middle ear 304.

FIG. 4 shows an example of an embodiment of a drug delivery device 400 having an internal supporting wire 402, which acts to provide structural stiffening to provide sufficient rigidity to the drug delivery device 400. In the embodiment shown the internal supporting wire 402 is wave-shaped, but which in other embodiments it may have other shapes, such as straight. One or more stabilizing holding wings 405 of the same polymer material as the rest of the drug delivery device 400 protrude from the extra-cochlear portion 403 to provide additional structural stability and aid in establishing a fixed position to the inserted device. A tapering section 406 occludes the opening in the round window membrane where the intra-cochlear portion 401 penetrates into the inner ear. Colored marking rings 407 around the intra-cochlear portion 401 give gradated information on the insertion depth of the device.

In the embodiment shown in FIG. 4, the internal supporting wire 402 extends from the extra-cochlear portion 403 to the intra-cochlear portion 401 and terminates in a sharp metallic puncturing point 404 towards the apical end of the intra-cochlear portion 401 for puncturing the round window membrane to insert the intra-cochlear portion 401 into the inner ear. In specific embodiments, this puncturing point 404 may be in the specific form of a coring or non-coring needle point. Assembling a beveled puncturing point 404 to a sufficiently flexible drug delivery device 400 can facilitate introduction of the device into the scala tympani in a single surgical move.

FIG. 5 shows a partial cross-section of the intra-cochlear portion 501 of another embodiment of a drug delivery device 500 having a drug eluting silicone polymer material for time released delivery of a therapeutic drug. Metallic rod 502 provides mechanical stability and terminates in a sharp metallic tip 504.

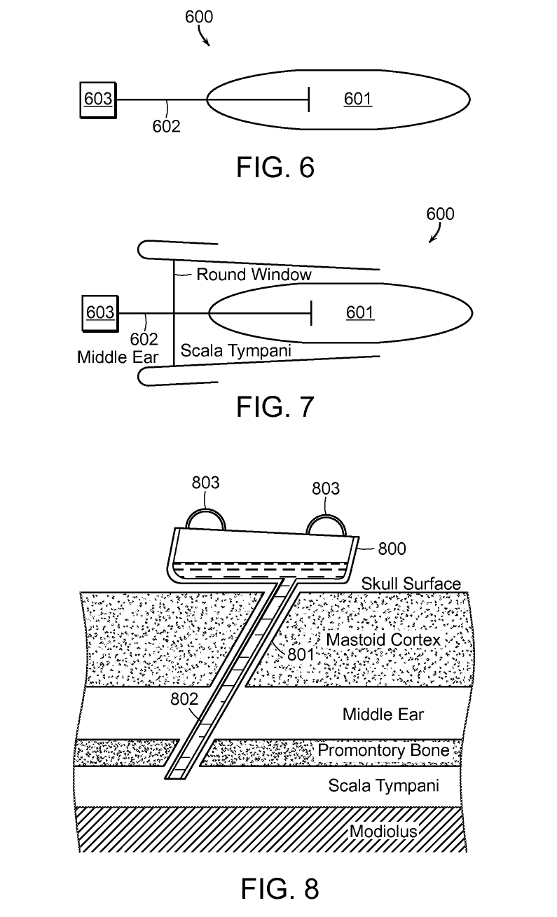

FIG. 6 shows an example of an embodiment of another drug delivery device 600 where the intra-cochlear portion 601 has opposing conical ends. The conical-shaped ends of the intra-cochlear portion 601 facilitate insertion through a slit in the round window membrane as shown in FIG. 7. In addition the intra-cochlear portion 601 has an anchored rod 602 in the interior of the drug delivery material, which extends out through the round window membrane into the middle ear. This allows healing of the penetration slit in the round window membrane slit the drug delivery material in the intra-cochlear portion 601 has been inserted completely into the scala tympani of the inner ear. Closure of the round window membrane is easier and faster around the thin rod of the anchor rod 602 instead of the larger diameter intra-cochlear portion 601 of the drug delivery member 600. The smaller diameter rod shape of the anchor rod 602 also minimizes interference with the normal sound-induced vibration of the round window membrane. The end of the anchor rod 602 extending into the middle ear includes a retrieval knob 603 for pulling the drug delivery device 600 out of the cochlea as may sometimes be useful for replacing the drug delivery material or dealing with an infection. Retrieval of the drug delivery device 600 can occur after re-slitting the round window membrane around the anchor rod 602 which traverses the membrane. At that point, pulling back on the retrieval knob 603 allows the conical end of the intra-cochlear portion 601 to exit through the round window membrane toward the middle ear.

The techniques described herein also can be used as part of a drug delivery system for delivering a therapeutic drug to the middle ear or to the inner ear. FIG. 8 shows an embodiment of such a system where a drug reservoir 800 is implanted on the surface of the skull bone over a catheter passage 801 that passes through the mastoid cortex, the middle ear, an promontory bone directly into the cochlea scala. Instead of an implant electrode, a rigid catheter 802 delivers drug fluid from the drug reservoir 800 into the scala tympani. An embodiment may be designed for delivering a drug to the middle ear, in which case, the entire drug catheter 802 may be rigid. For drug delver into the inner ear such as the scala tympani, then the apical tip of the drug catheter 802 is softer and flexible, similar in mechanical properties to the implant electrode described above. One or more septum ports 803 on the drug reservoir 800 may allow for insertion of a syringe needle to refill the reservoir.

It is understood that lubricants, lubricious coating, anti inflammatory coating, may be used in combination with the device and accessories described here. It is also understood that the implant electrode, drug delivery catheter, and the various accessories may be beneficial if using some type of endoral surgical approach, canal wall drill out, etc.

Although various exemplary embodiments of the invention have been disclosed, it should be apparent to those skilled in the art that various changes and modifications can be made which will achieve some of the advantages of the invention without departing from the true scope of the invention.

What is claimed is:

1. A drug delivery device for the inner ear, comprising:
a drug delivery member without stimulation electrodes and including:
i. an intra-cochlear portion for penetrating into the inner ear of the patient and containing a drug eluting polymer material having at least one therapeutic drug for release over time in a therapeutically effective amount into fluid in the inner ear of the patient, and
ii. an extra-cochlear portion that resides in the middle ear of a patient and completely occludes where the intra-cochlear portion penetrates into the inner ear; and
an anchor rod within the intra-cochlear portion of the drug delivery member and adapted to extend out into the middle ear, ending in a retrieval knob for pulling the drug delivery member out of the cochlea.

2. A device according to claim 1, wherein the intra-cochlear portion is a cylindrical rod.

3. A device according to claim 1, wherein the intra-cochlear portion has a conical shape.

4. A device according to claim 1, further comprising:
a support wire within at least a portion of the drug delivery device to provide supporting stability to the drug delivery member.

5. A device according to claim 4, wherein the support wire includes a puncturing point towards an apical end of the intra-cochlear portion for puncturing the round window membrane to insert the intra-cochlear portion into the inner ear.

6. A device according to claim 1, wherein the anchor rod is adapted to penetrate through the round window membrane.

7. A device according to claim 1, wherein the intra-cochlear portion of the drug delivery member has opposing conical ends.
